(19) United States
(12) Patent Application Publication
(10) Pub. No.: US 2026/0130660 A1
(43) Pub. Date: May 14, 2026

(54) Device For Retraction Of Nostril During Nasal Procedures

(71) Applicant: Valent Medical Inc., Ridgefield, CT (US)

(72) Inventors: Yosef Krespi, Ridgefield, CT (US); Ron Hadani, Englewood, NJ (US)

(21) Appl. No.: 19/382,491

(22) Filed: Nov. 7, 2025

Related U.S. Application Data
(60) Provisional application No. 63/718,444, filed on Nov. 8, 2024.

(51) Int. Cl. A61B 17/02

(52) U.S. Cl. CPC A61B 17/0206; A61B 2017/0287

(57) ABSTRACT

There is provided a nostril retracting device which includes a monolithic shape changing and flexible frame which has a pair of retracting arms bound to the perimeter of the frame, with the frame having a pair of side wings and a central area section. The retracting arms extend upward and away from the frame at an angle, with each of the retracting arms culminating in a retracting prong at a distal end of the arm. The frame includes an opening for receiving a pin or screw through the frame and adjustment means connected at a proximal end of the pin or screw. By moving the pin or screw through the frame to contact and apply force to the central area section of the frame and moving it upward, the wings and retracting arms spread apart. The adjustment means are used to return the frame to the original position.

### CROSS REFERENCE TO RELATED APPLICATIONS

This application claims benefit of and priority to U.S. Provisional Application Ser. No. 63/718,444 filed on Nov. 8, 2024 and incorporated herein by reference in its entirety.

### FIELD OF THE INVENTION

The present invention relates to a retraction device for opening the nostril area, and more particularly a retraction device used in treatment of the nasal valve area.

### BACKGROUND

For medical procedures in the nasal valve and nostril area, a common issue is obtaining proper visibility for the medical professional in the area of interest for treatment. Additionally, the nostril area is limited for receiving medical instruments for a procedure. In order for proper and improved medical procedures, there is a need to maintain the nostril retracted and opened for the duration of the procedure. A need also exists to expose the nasal valve area for potential medical procedures which are not capable at present due to limited visibility, access, and limited maneuverability of the medical tools.

### SUMMARY OF THE INVENTION

The present invention is a nostril retracting device which includes a monolithic shape-changing and flexible frame. The monolithic shape-changing and flexible frame has a pair of side wings and a central area section, with a pair of retracting arms extending upward and away from the side wings of the monolithic flexible frame at an angle. Each of the retracting arms has a nostril latching retracting prong at the distal end of each of the respective retracting arms. The proximal end of the frame includes an opening for receiving a pin or screw through the frame. The opening includes an adjustment means connected at the proximal end of the pin/screw for moving the pin/screw through the frame. The distal end of the pin/screw is in contact with the proximal end of the central area section of the frame. When moving upward the pin/screw moves the central area section of the frame upward as force is applied to the proximal end of the central area section by the upward moving pin/screw. The upward movement of the central area section of the frame which is caused by the forward movement of the pin/screw causes the pair of the side wings of the frame to move outward. The nostril retracting device of the present invention also includes an embodiment where the adjustment means includes a knob for moving the pin or screw. The knob may be rotated manually by the medical professional to move the pin/screw upward and apply pressure on the proximal end of the central area section of the frame, which causes it to move upward and as a result the monolithic flexible frame's side wings with the pair of retracting arms are moving outward and increase the level of retraction. By returning the knob in the opposite direction, the pin/screw is returned backwards towards its original position, the pressure on the proximal end of the central area of the frame is reduced, the central area of the frame moves backwards, and the pair of the side wings of the frame, together with the pair of retracting arms are moving inward, reducing the level of retraction. The monolithic flexible frame thereby returns to its original position.

In an embodiment, the nostril retracting device of the present invention is a U-shaped frame. In an embodiment, the nostril retracting device according to the present invention has the pair of retracting arms extending away from the frame at an offset angle of 20 degrees to adhere to the contour of a patient's face when the device is in use.

The nostril retracting device of the invention includes an embodiment where the retracting arms are received in a side slot along the frame. In an embodiment, the nostril retracting device of the present invention includes where the pair of retracting arms are made of semi-rigid or a spring wire.

In an embodiment, the nostril retracting device of the present invention includes where the frame has a perimeter and the pair of retracting arms are connected to the frame along the perimeter of the frame, preferably the outer perimeter of the frame.

In an embodiment, the nostril retracting device of the present invention includes where the pin is a screw or a bolt.

In an embodiment, the nostril retracting device of the present invention includes where the retracting prong of each of the retracting arms has a predetermined bend for adhering to a nostril wall of a patient when in use. In use, the retracting prongs are 5 mm apart in a closed position and are in a range of 5 mm up to 35 mm apart in an open or retracted position.

The monolithic frame of the nostril retracting device of the present invention may be constructed of or formed from polymer or other flexible and elastic material.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below where:

FIG. 1 is an illustration of the device of the present invention.

FIG. 2 is an illustration of the device of the present invention in use with a patient.

FIG. 3 is an illustration of the present invention in a closed position.

FIG. 4 is an illustration of the present invention in the retracted position.

FIG. 5 is an illustration of the present invention in a closed position with dimensions.

FIG. 6 is an illustration of the device in a retracted position with dimensions.

FIG. 7 is a side view of the device of the present invention.

### DETAILED DESCRIPTION

The present invention is a device for retracting nostrils of a patient, useful during nasal procedures and treatment of nasal valve area, such as allowing improved exposure and access for a laser to treat the front of the nose and nasal valve area.

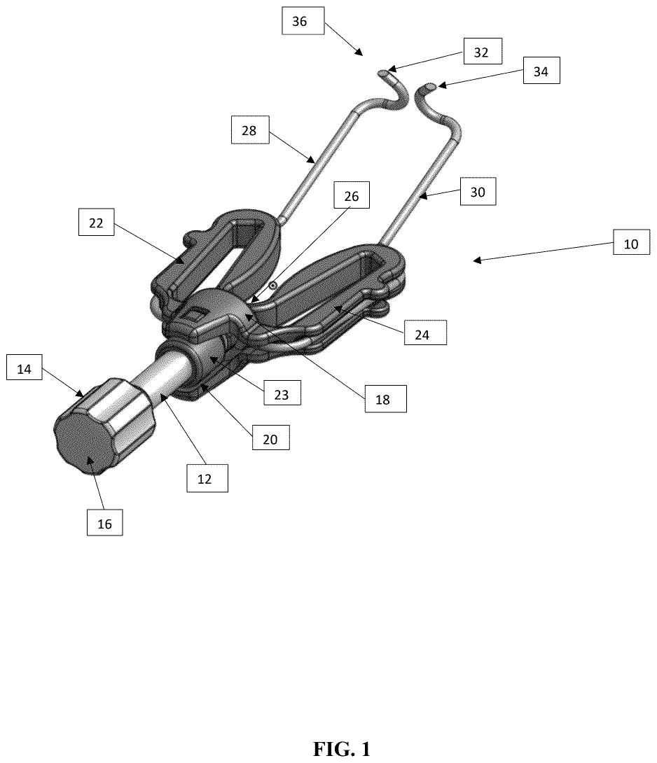

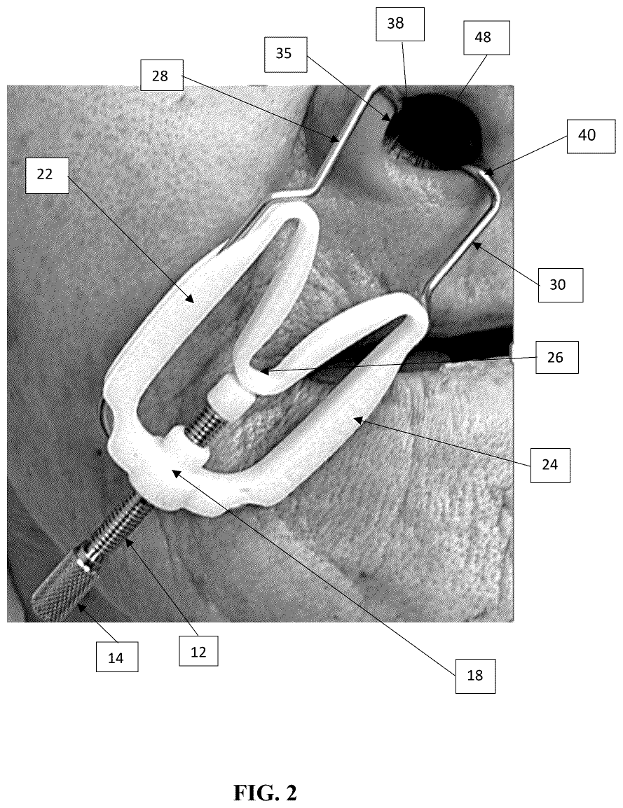

The present invention includes the exposure of the internal nasal valve using an adjustable and self-retaining nasal valve retractor. Referring to FIG. 1 and FIG. 2, there is shown the retractor of the present invention 10 and its use in a patient for the present invention (FIG. 2). The nostril and nasal valve retractor 10 has a pair of distal end retracting prongs 32, 34 which are controlled at the proximal end by an adjustment knob 14 or other similar mechanical adjustment means, such as clamps, handles, springs or tensions control mechanisms. By turning the adjustment knob 14, the medical professional is able to open and close the distal prongs 32, 34 during a procedure.

As shown in FIG. 1, the retractor device 10 has a shape changing, flexible monolithic frame 18 with a pair of retracting wire arms 28, 30 attached to or bound around the perimeter of the frame 18. The frame 18 is of a general U-shape, although other shapes are within the scope of the invention, with a left side wing 22, right side wing 24 and a central area 26 of the frame 18. The retracting wire arms 28 and 30 extend upward and away from the frame 18 at a slight raised angle and culminate in respective prongs 32 and 34 at the distal end 36 for each retracting arm 28, 30. The frame 18 is connected to a screw or pin 12 which has an adjustable handle or knob 14 at the proximal end 16. The screw or pin 12 is received by the frame 18 at an opening 20 in the frame 18 which allows for the screw or pin 12 to be secured inside the frame 18, such by mating threads inside the opening of the frame or alternative means, such as a snug-fit plug 23. By adjusting or rotating the knob 14, the screw or pin 12 is moved upward through the opening 20 in order to push the frame 18 to an open position, as distinguished from the closed position shown in FIG. 1. By monolithic, the term includes a single unitary piece and solid structure frame.

With respect to the frame 18, in an embodiment, the general U-shape of the frame has rounded corners at the top of each of its side wings 22, 24. In an embodiment, the frame 18 has a slot or groove along the outer surface of the frame for receiving the wire retracting arms 28, 30.

The retractor 10 is shown again in FIG. 2 with the distal bends 38 and 40 at the distal end of the retracting wire arms 28 and 30 that adhere to the contour of the nostril wall inserted into a nostril 48 of a patient. The retractor prongs 32, 34 are adjusted in order to open up the nostril 48 and maintain the nostril 48 in a widened position so that the nasal cavity is more exposed for the medical professional. For a procedure, such as an ablation, a handheld laser device with fiber tip is inserted through the nostril and to the nasal valve area for ablating fatty and cartilaginous tissue for opening the nasal pathway and improving airway patency.

As seen in the view of the nostril 48 in FIG. 2, the retractor prongs 32,34 are placed inside the edges of the nasal wall 35 so that the nostril 48 can be expanded. The medical professional can use a laser handheld device (not shown) so that a laser fiber tip is inserted into the nostril 48 as it is held open by the retractor 10. The tissue in the nasal cavity at a desired treatment location may then be ablated by the laser fiber tip in either non-contact, interstitial or contact mode.

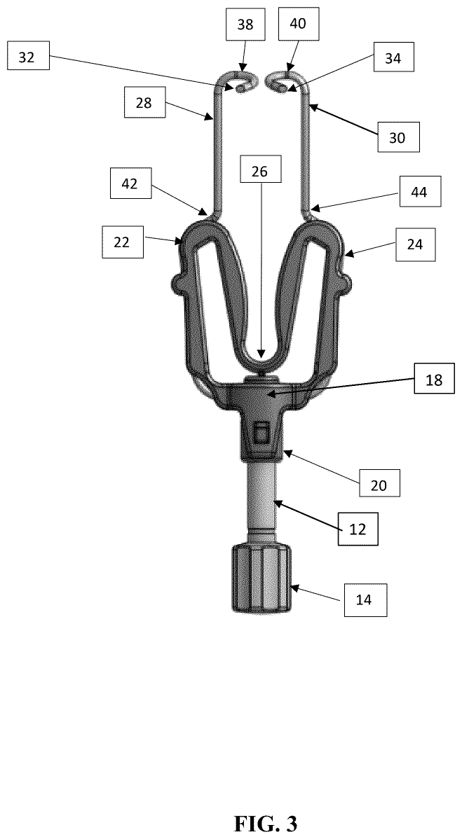

As seen in FIG. 3, the monolithic nasal retractor 10 of the present invention is shown in the closed position. The screw 12 applies vertical force upon the central area 26 which deforms the monolithic frame 18. When rotated counterclockwise, the screw 12 moves upward in the opening 20 and pushes the central area 26 of the monolithic frame 18 forward, causing its U-shape to widen so that the left side wing 22 and right side wing 24 separate from each other and move outward. In this manner, the retracting wire arms 28 and 30 are opened.

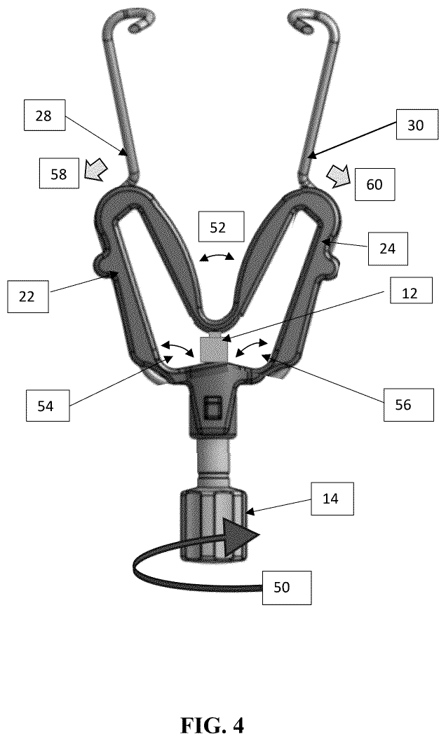

FIG. 4 illustrates the monolithic frame 18 in the opened position. The handle/knob 14 is turned, as indicated by arrow 50, and pushes the screw 12 upward where it applies vertical force on the inner central area 26 of the frame 18 to deform it. This opens the frame 18 at the central area 26, as indicated by arrow 52, which deforms the frame as indicated by arrows 54 and 56 and pushes the left and right wings 22, 24 outward and spread apart as indicated by arrows 58, 60. At the top of the frame 18, the wire matches and aligns with the circumference and perimeter of the frame 18 at each of the left and right wings 22, 24 and then has a bend 42 and 44 upward and angled away from the frame 18 to form the respective retracting wire arms 28 and 30. As the wings 22, 24 are spread apart at the top (indicated by arrows 58, 60), the retracting wire arms 28 and 30 are moved apart from each other to an open or the retracted position. By turning the knob 14 in the opposite direction, the screw 12 is moving downward, pulling with it the inner central area 26 of the frame 18, and the upward deforming force is removed. This allows the frame 18 to return to its prior (steady-state) shape.

Preferably, the retracting wire arms 28 and 30 are formed of a single stainless steel wire (e.g., 304 or 17-7 type) which are a single piece looped around the frame 18 in a groove. As the wire is looped around the circumference and perimeter of the monolithic frame 18, by opening the frame 18, the retracting wire arms 28 and 30 are separated as well. At the distal end of each of the retracting wire arms 28 and 30 there is a specially designed predetermined bend 38 and 40 that adheres to the contour of the nostril wall. Each of the bend 38 and 40 is terminated with the prongs 32 and 34 to secure the retracting wire arms to the nostril's inner tissue and prevent the retracting wire arms from sliding over the mucosa of the nostril when in use on a patient.

As the force is applied by the turning of the screw 12, the frame 18 exhibits flexibility and elasticity. In an embodiment, this is due to the frame 18 preferably made of a polymer, such as polyethylene, ABS, etc., although other materials with flexibility and elasticity may be used.

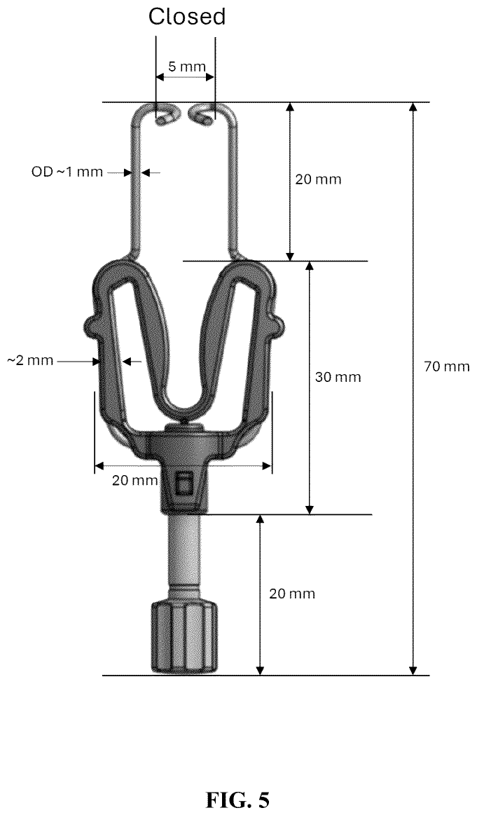

Referring to FIG. 5, there is shown the present invention device 10 in the closed position with indicated dimensions for the material elements of an embodiment of the invention, although other dimensions are within the scope. Overall, the device is 70 mm long from the proximal end 16 to the distal end 36. In the closed position, the prongs 32 and 34 at the distal end 36 are separated by 5 mm. The wire has an outside diameter of 1 mm and the retracting wire arms 28 and 30 extend 20 mm above the frame 18. The frame 18 has a length of 30 mm from the top of the frame to the bottom and has a width about of 20 mm. The frame 18 has a width of approximately 2 mm. The screw 12 has a length of approximately 20 mm from the proximal end 16 to the opening 20 on the frame 18.

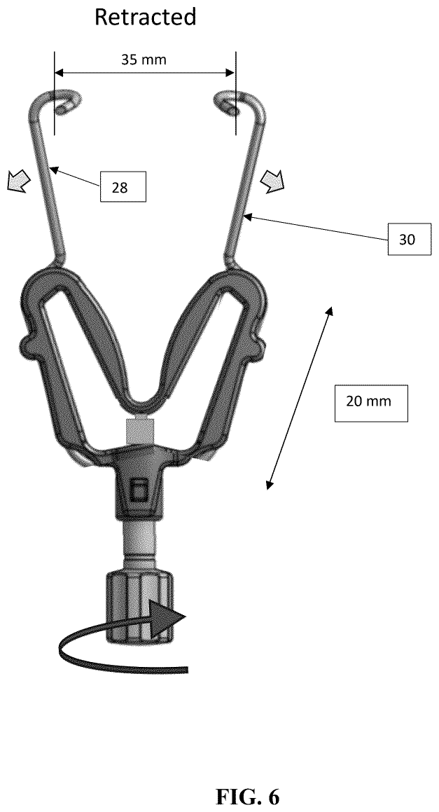

Referring to FIG. 6, there is shown the device of the present invention with the retracting wire arms 28 and 30 separated and in a position for retracting the nostril. By turning the handle/knob 14 and moving the screw or pin upward, the monolithic frame 18 deforms and opens. The respective prongs 32 and 34 at the distal end 36 of the retracting wire arms 28 and 30 are now separated from the initial closed position in a range of 5 mm up to approximately 35 mm. The side wings of the frame 22 and 24 have a length of approximately 20 mm.

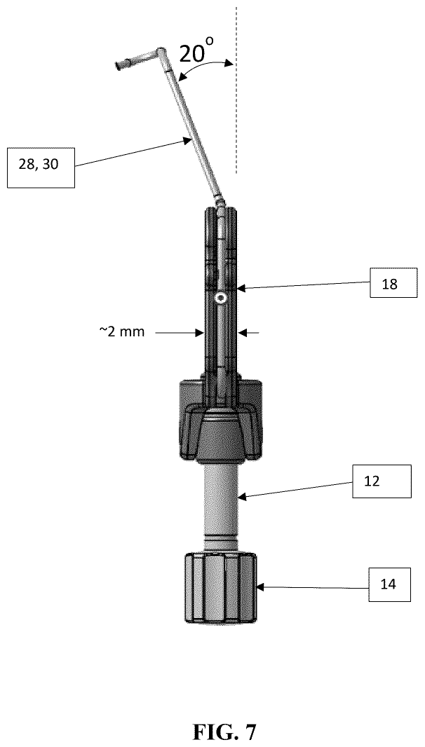

In FIG. 7, there is shown a side view of the retractor device 10 of the present invention. In this view, it is shown that the retracting wire arms 28 and 30 extend from the frame 18 at an angle of approximately 20 degrees. This enables the device to adhere to the contour of the face of the patient when the device is in use. The thickness of the frame 18 is indicated as 2 mm in a non-limiting example.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A nostril retracting device comprising:
a monolithic shape-changing and flexible frame, said frame having a pair of retracting arms connected to said frame;
said frame having a pair of side wings and a central area section;
said retracting arms extending upward and away from said frame at an angle;
each of said retracting arms having a bend and a prong at a distal end of each of said arms;
said frame having an opening in said frame for receiving a pin through said frame;
adjustment means connected at proximal end of said pin for moving said pin through said frame to contact said central area section of said frame and move said central area section of said frame upward as force is applied to said central area section by said pin.

2. The nostril retracting device according to claim 1 wherein said frame is U-shaped.

3. The nostril retracting device according to claim 1 wherein said arms are received in a slot along said frame.

4. The nostril retracting device according to claim 1 wherein said angle of the retracting arms is 20 degrees offset from said frame.

5. The nostril retracting device according to claim 1 wherein said pair of retracting arms are made of wire.

6. The nostril retracting device according to claim 1 wherein said pair of retracting arms are made of spring.

7. The nostril retracting device according to claim 1 wherein said frame has a perimeter and said pair of retracting arms are connected to said frame along said perimeter of said frame.

8. The nostril retracting device according to claim 1 wherein said pin is a screw.

9. The nostril retracting device according to claim 1 wherein each said retracting arm has a predetermined bend for adhering to a nostril opening and a retracting prong.

10. The nostril retracting device according to claim 1 wherein said retracting prongs are 5 mm apart in a closed position.

11. The nostril retracting device according to claim 1 wherein said retracting prongs are in a range of 5 mm up to 35 mm apart in an open position.

12. The nostril retracting device according to claim 1 wherein said adjustment means includes a knob for moving said pin.

13. The nostril retracting device according to claim 1 wherein said frame is formed of a polymer.

14. The nostril retracting device according to claim 1 wherein said pair of side wings move outward upon said pin contacting said central area section of said frame and pushing said central area section of said frame upward.
